(19) United States
(12) Patent Application Publication
(10) Pub. No.: US 2006/0149380 A1
(43) Pub. Date: Jul. 6, 2006

(54) Systems, devices and methods for treatment of intervertebral disorders

(76) Inventors: Jeffrey C. Lotz, San Mateo, CA (US); David S. Bradford, Sausalito, CA (US)

Correspondence Address: JOHN P. O'BANION;O'BANION & RITCHEY LLP, 400 CAPITOL MALL SUITE 1550, SACRAMENTO, CA, 95814, US

(21) Appl. No.: 11/292,335

(22) Filed: Dec. 1, 2005

Related U.S. Application Data
(60) Provisional application No. 60/632,396, filed on Dec. 1, 2004.

(51) Int. Cl. A61F 2/44

(52) U.S. Cl. 623/17.12; 623/17.11; 623/17.16

(57) ABSTRACT

A bioactive/biodegradable nucleus implant for repairing degenerated intervertebral discs that is inflated inside the nucleus space after the degenerated nucleus has been removed to re-pressurize the nuclear space within the intervertebral disc. The implant is inflated with a high molecular weight fluid, gel or combination of fluid and elastomer, preferably an under-hydrated HA hydrogel/growth factor mixture with or without host cells. The implant includes an internal, integral, self-sealing valve that allows one-way filling of the implant after it is placed within the disc, and is made from a material that allows fibrous in growth thereby stabilizing the implant. A variety of substances can be incorporated into the implant to promote healing, prevent infection, or arrest pain.

### CROSS-REFERENCE TO RELATED APPLICATIONS

This application claims priority from U.S. provisional application Ser. No. 60/632,396 filed on Dec. 1, 2004, incorporated herein by reference in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISC

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. § 1.14.

### BACKGROUND OF THE INVENTION

1. Field of the Invention

The present invention pertains generally to repairing intervertebral disc disorders, and more particularly to implants and surgical procedures for repairing a degenerated intervertebral disc.

2. Description of the Background Art

An estimated 4.1 million Americans annually report intervertebral disc disorders, with a significant portion of them adding to the nearly 5.2 million low-back disabled. Though the origin of low-back pain is varied, the intervertebral disc is thought to be a primary source in many cases, and is an initiating factor in others where a degenerated disc has led to altered spinal mechanics and non-physiologic stress in surrounding tissues.

The intervertebral disc is a complex structure consisting of three distinct parts: the nucleus pulposus; the annulus fibrosus; and the cartilaginous end-plates. The nucleus pulposus is a viscous, mucoprotein gel that is approximately centrally located within the disc. It consists of abundant sulfated glycosaxninoglycans in a loose network of type II collagen, with a water content that is highest at birth (approximately 80%) and decreases with age. The annulus fibrosus is that portion of the disc which becomes differentiated from the periphery of the nucleus and forms the outer boundary of the disc. The transition between the nucleus and the annulus is progressively more indefinite with age. The annulus is made up of coarse type I collagen fibers oriented obliquely and arranged in lamellae which attach the adjacent vertebral bodies. The fibers run the same direction within a given lamella but opposite to those in adjacent lamellae. The collagen content of the disc steadily increases from the center of the nucleus to the outer layers of the annulus, where collagen reaches 70% or more of the dry weight. Type I and II collagen are distributed radially in opposing concentration gradients. The cartilaginous end-plates cover the end surfaces of the vertebral bodies and serve as the cranial and caudal surfaces of the intervertebral disc. They are composed predominately of hyaline cartilage.

The disc derives its structural properties largely through its ability to attract and retain water. The proteoglycans of the nucleus attract water osmotically, exerting a swelling pressure that enables the disc to support spinal compressive loads. The pressurized nucleus also creates tensile pre-stress within the annulus and ligamentous structures surrounding the disc. In other words, although the disc principally supports compressive loads, the fibers of the annulus experience significant tension. As a result, the annular architecture is consistent with current remodeling theories, where the ˜60° orientation of the collagen fibers, relative to the longitudinal axis of the spine, is optimally arranged to support the tensile stresses developed within a pressurized cylinder. This tissue pre-stress contributes significantly to the normal kinematics and mechanical response of the spine.

When the physical stress placed on the spine exceeds the nuclear swelling pressure, water is expressed from the disc, principally through the semipermeable cartilaginous end-plates. Consequently, significant disc water loss can occur over the course of a day due to activities of daily living. For example, the average diurnal variation in human stature is about 19 mm, which is mostly attributable to changes in disc height. This change in stature corresponds to a change of about 1.5 mm in the height of each lumbar disc. Using cadaveric spines, researchers have demonstrated that under sustained loading, intervertebral discs lose height, bulge more, and become stiffer in compression and more flexible in bending. Loss of nuclear water also dramatically affects the load distribution internal to the disc. In a healthy disc under compressive loading, compressive stress is created mainly within the nucleus pulposus, with the annulus acting primarily in tension. Studies show that, after three hours of compressive loading, there is a significant change in the pressure distribution, with the highest compressive stress occurring in the posterior annulus. Similar pressure distributions have been noted in degenerated and denucleated discs as well. This reversal in the state of annular stress, from physiologic tension due to circumferential hoop stress, to non-physiologic axial compression, is also noted in other experimental, analytic and anatomic studies, and clearly demonstrates that nuclear dehydration significantly alters stress distributions within the disc as well as its biomechanical response to loading.

The most consistent chemical change observed with degeneration is loss of proteoglycan and concomitant loss of water. This dehydration of the disc leads to loss of disc height. In addition, in humans there is an increase in the ratio of keratan sulphate to chondroitin sulphate, an increase in proteoglycan extractability, and a decrease in proteoglycan aggregation through interaction with hyaluronic acid (although the hyaluronic acid content is typically in excess of that needed for maximum aggregation). Structural studies suggest that the non-aggregable proteoglycans lack a hyaluronate binding site, presumably because of enzytruitic scission of the core protein by stromelysin, an enzyme which is thought to play a major role in extracellular matrix degeneration. These proteoglycan changes are thought to precede the morphological reorganization usually attributed to degeneration. Secondary changes in the annulus include fibrocartilage production with disorganization of the lamellar architecture and increases in type II collagen.

Currently, there are few clinical options to offer to patients suffering from these conditions. These clinical options are all empirically based and include (1) conservative therapy with physical rehabilitation and (2) surgical intervention with possible disc removal and spinal fusion. In contrast to other joints, such as the hip and knee, very few methods of repair with restoration of function are not available for the spine.

Therefore, there is a need for a minimally invasive treatment for degenerated discs which can repair and regenerate the disc. The present invention satisfies that need, as well as others, and overcomes the deficiencies associated with conventional implants and treatment methods.

### BRIEF SUMMARY OF THE INVENTION

The present invention comprises an implant and minimally invasive method of treating degenerated discs which can repair and regenerate the disc. More particularly, the present invention comprises a bioactive/biodegradable nucleus implant and method of use. The implant is inflated inside the nucleus space after the degenerated nucleus has been removed to re-pressurize the nuclear space within the intervertebral disc. Nuclear pressure produces tension in the annular ligament that increases biomechanical stability and diminishes hydrostatic tissue pressure that can stimulate fibro-chondrocytes to produce inflammatory factors. The device will also increase disc height, separate the vertebral bodies and open the spinal foramina.

By way of example, and not of limitation, an implant according to the invention comprises a collapsible, textured or smooth membrane that forms an inflatable balloon or sack. To inflate the implant, the implant is filled with a high molecular weight fluid, gel or combination of fluid and elastomer, preferably an under-hydrated HA hydrogel/growth factor mixture with or without host cells. Integral to the membrane is a self-sealing valve that allows one-way filling of the implant after it is placed within the disc. The implant membrane is made from a material that allows fibrous in-growth thereby stabilizing the implant. A variety of substances can be incorporated into the device to promote healing, prevent infection, or arrest pain. The implant is inserted utilizing known microinvasive technology. Following partial or total nucleotomy with a small incision, typically annular, the deflated implant is inserted into the nuclear space through a cannula. The implant is then filled through a stem attached to the self-sealing valve. Once the implant is filled to the proper size and pressure, the cannula is removed and the annular defect is sealed.

One of the main difficulties in repairing the degenerated disc is increasing the disc height. The disc and surrounding tissues such as ligaments provide a great deal of resistance to disc heightening. For this reason it is unlikely that placing a hydrogel alone into the nuclear space will be able to generate enough swelling pressure to regain significant disc height. The present invention, however, addresses this problem by allowing initial high pressures to be generated when the implant is inflated in the nuclear space. The initial high pressure is sufficient to initiate the restoration of the original disc height. This initial boost in disc height facilitates the later regeneration stages of this treatment.

In the long term, having a permanent pressurized implant is not likely to be ideal because it may not be able to mimic the essential biomechanical properties of the normal disc. However, the invention also addresses this issue by using a biodegradable sack. The initially impermeable membrane permits high pressurization. When the membrane biodegrades, it allows the hydrogel mixture to take action in playing the role of the normal nucleus pulposus with its inherent swelling pressure and similar mechanical properties.

A variety of growth factors or other bioactive agents can be attached to the surface of the implant or included in the hydrogel mixture that is injected inside the implant. The membrane could be reinforced or not reinforced with a variety of fiber meshes if necessary. Furthermore, a variety of materials could be used for the membrane; the only requirement is that they be biodegradable such that the membrane is impermeable when initially implanted and until it biodegrades. A variety of materials could be injected into the sack such as cartilage cells, alginate gel, and growth factors.

The present invention comprises systems, devices and methods, which can be employed alone or in any combination with each other or in any combination with systems, methods and devices known in the art, in connection with treatment of intervertebral disorders.

Another aspect of the invention is a stent for facilitating regeneration of an intervertebral nucleus and/or retention of a bladder-type implant, wherein the intervertebral nucleus is bounded at its upper and lower extremities by opposing vertebral endplates of adjacent vertebrae, and at its periphery by annulus fibrosus. The stent has top and bottom portions comprising metal hoops having a footprint adapted to engage with peripheral regions of the opposing vertebral endplates while leaving a central region of the vertebral endplates open. The stent also includes a plurality of lateral members connecting said top and bottom portions. The lateral members and top and bottom portions are configured to allow the stent to collapse for insertion into the nuclear cavity via an annulus port and then expand upon placement in the nuclear cavity.

In some embodiments, where the stent is configured to be installed in between adjacent lumbar vertebrae, the top and bottom hoops may have an increased ring gauge to accommodate higher compressive loads.

In an alternative embodiment, the stent is configured to be installed in between adjacent cervical endplates. Accordingly, the stent may extend across the majority of the vertebral endplates outward through the region normally occupied by the annulus. In this configuration the upper and lower hoops are preferably elliptical to match the contours of the vertebral bodies. Furthermore, the upper and lower hoops may have a series of serrations to engage the vertebral bodies. The hoops may also have one or more flanges that extend to the anterior portions of the outside wall of the vertebral body, thereby allowing fixation to the anterior surfaces of the vertebra.

In some modes of the present aspect, the stent is configured to support at least a portion of compression loads generated between the opposing vertebral endplates to facilitate regeneration of the intervertebral nucleus. In some embodiments, the stent functions as a flexible cage to allow movement of the vertebral endplates while at the same time keeping the nuclear cavity open for tissue regeneration. The footprint of the top and bottom portions may be circular, or somewhat elliptical to match the anatomy of the intervertebral nucleus.

Preferably, the metal hoops and lateral members comprise a memory material, such as nitinol. The hoops may also be textured and/or a growth factor to promote bony in growth, or an anti-inflammatory factor to treat discogenic pain.

In an alternative embodiment, the stent is configured to be expanded around an inflatable membrane. In this case, the inflated membrane supports intervertebral compression, while the stent prevents membrane lateral expansion or lateral migration.

Yet another aspect of the invention is a method for facilitating regeneration of the intervertebral disc, comprising inserting a collapsed stent into a nuclear cavity in the nucleus pulposus tissue, and expanding the stent to support a portion of intervertebral compression loads and thereby facilitate nuclear regeneration.

In a preferred mode, inserting the collapsed stent is done by creating an annular portal annulus fibrosus to access the nucleus pulposus, removing the nucleus pulposus tissue to create the nuclear cavity, and inserting the collapsed stent through the annular portal and into the nuclear cavity. In the cervical spine, most of the anterior and posterior annulus is removed prior to stent placement, and in this case, implant retention is facilitated by anterior flanges.

Generally, the upper and lower metal hoops to are expanded to engage the vertebral endplates, and generate an axial force on the vertebral endplates via a loading from the plurality of lateral members to separate the upper and lower hoops against the endplates.

In an another embodiment, an inflatable membrane may be first inserted into a nuclear cavity in the nucleus pulposus tissue, and then the inflatable membrane is expanded to further support a portion of intervertebral compression loads and thereby facilitate nuclear regeneration. Alternatively, the stent is inserted into a nuclear cavity while in a collapsed configuration over the inflatable membrane, and inflation of the inflatable membrane releases the stent from the collapsed configuration.

Yet another aspect of the invention is an implant for repairing an intervertebral disc. The implant has an inflatable membrane with an inner layer configured to withstand compressive forces generated in the intervertebral disc, and a textured external layer that to promotes fibrous tissue in growth in the intervertebral disc.

In some embodiments, the textured layer is formed from a foamed, uncured polyurethane. An exemplary textured layer may have an average pore size ranging from approximately 400 microns to approximately 800 microns, and a volume porosity in the range of approximately 75% to approximately 80%.

The implant may also have an internal self-sealing fill valve for filling the membrane. In some embodiments, the valve comprises internal opposing walls that collapse as a result of a compressive load disposed on said internal chamber.

A further aspect of the invention is a method for creating a textured inflatable implant by forming an inflatable membrane, and dipping the inflatable membrane into a solution of foamed, uncured polyurethane to form a final textured surface layer.

Yet another aspect of the invention is an implant having an inflatable membrane, a filler material comprising a first fluid for inflating the membrane, and a plurality of microspheres dispersed in said filler material, each of said microspheres holding a second fluid. The microspheres may be filled with gas, or with a liquid to help maintain hydration of the first fluid over a period of time.

The microspheres may also be configured to promote movement of fluid between the microspheres and the first fluid based on pressure exerted on the first fluid. For example, the microspheres may transfer the second fluid to the first fluid at rate that increases with increased pressure. The second fluid inside the microspheres may be water, therapeutic agent, or other solution beneficial in promoting healing.

Yet a further aspect of the invention is an implant for repairing an intervertebral disc disposed between opposing vertebral endplates of adjacent vertebrae. The implant has membrane having upper and lower walls configured to engage said vertebral endplates, and reinforced peripheral walls joining the upper and lower walls. The peripherally reinforced walls may have a variety of beneficial attributes, including prevent bulging of the membrane a result of compressive forces imposed on said membrane from the vertebral endplates, increasing fatigue resistance, or providing stiffness in an under inflation condition. Additionally, the reinforced peripheral wall may create a nonlinearity in overall device stiffness during bending or compression to improve overall intervertebral stability

In one embodiment, the peripheral walls are thicker than the upper and lower walls have to provide localized stiffness. As an alternative or addition, the peripheral walls may also be reinforced with a fiber matrix. For example, the fiber matrix comprises a plurality of woven fibers oriented at an angle of approximately 60 degrees relative to vertical.

Yet another aspect is an implant comprising membrane with a plurality of inner chambers for holding an inflation medium.

In one embodiment, the membrane has a first chamber with a different stiffness than the second chamber. For example, the first chamber may be filled with a gel having a first stiffness, and the second chamber may be filled with a gel having a second stiffness that is stiffer than the first gel. The second chamber may also surround the periphery of the first chamber.

Preferably, the first chamber and the second chamber have independent, concentrically oriented valves.

In another embodiment, wherein the first chamber is configured to hold a gel to mechanically support the opposing vertebral endplates, with the second chamber holding a therapeutic agent to promote tissue in growth.

Another aspect is a method of treating a region of annulus fibrosus disposed between adjacent vertebral bodies. The method includes the steps of installing one or more sutures into a vertebral body rim adjacent to the annulus fibrosus region, attaching the one or more sutures to a netting, and securing the netting across the annulus fibrosus region.

Preferably, the netting is secured across an annulus defect, such as hole in the annulus or annulus degeneration. In addition the netting may have one side (the side away from the annulus) with an anti-adhesion film to prevent connective tissue attachment. Accordingly, the side adjacent to the annulus would have an adhesion promoting surface that may consist of texture plus growth factor.

Preferably, at least two sutures are installed into the vertebral rim. The sutures may be installed simultaneously with use of a specially modified tool.

In one embodiment, the suture anchors are placed with a pliers-type tool with a plurality of tangs on each side, wherein each tang is adapted to attach to a suture anchor.

The sutures may be attached directly to the vertebral rim, or attached via installing suture anchors in the vertebral endplate adjacent to the annulus fibrosus region.

Yet a further aspect is a system for treating a region of annulus fibrosus having one or more anchors configured to be installed in the rim of each vertebral body, a netting configured to disposed across the annulus fibrosus region, and one or more sutures configured to attach the netting to the anchors. The netting preferably comprises a woven mesh. In some embodiments, woven mesh has a cross-ply matching the annulus fibrosus architecture. Additionally, one side of the mesh may have a polymer configured to promote tissue in growth, and an opposing side configured to prevent adhesion.

### BRIEF DESCRIPTION OF THE SEVERAL VIEWS OF THE DRAWING(S)

The invention will be more fully understood by reference to the following drawings which are for illustrative purposes only:

FIG. 1 is a side view of an implant according to the present invention, shown in a collapsed state.

FIG. 2 is a side view of the implant of FIG. 1, shown in an inflated state, with a portion of the membrane cut away to show the internal filler material.

FIG. 3 is cross-sectional side view of the implant of FIG. 1, shown in the inflated state and showing the integral, internal fill valve.

FIG. 4 is a side view of a mandrel for molding an implant according to the present invention.

FIG. 5 is a side view of an implant membrane according to the present invention as it would be seen after being dip molded on the mandrel shown in FIG. 4 but before removal from the mandrel.

FIG. 6 is an end view of the implant shown in FIG. 5 prior to heat-sealing the open end.

FIG. 7 is an end view of the implant shown in FIG. 5 after heat-sealing the open end.

FIG. 8 is an exploded view of a delivery system for placement of an implant according to the invention shown in relation to the implant.

FIG. 9 is an assembled view of the delivery stem shown in FIG. 8 with the implant attached.

FIG. 10 is a side schematic view of a degenerated intervertebral disc prior to repair using an implant according to the present invention.

FIG. 11A through FIG. 11G is a flow diagram showing a surgical procedure for placement of an implant according to the present invention.

FIG. 12 is a perspective view of an introducer sheath according to the invention with a trocar inserted and positioned in the nuclear space of an intervertebral disc so as to create an annular opening in the disc.

FIG. 13 is a perspective view of a Crawford needle and Spine Wand inserted in the introducer sheath shown in FIG. 12 and positioned for ablation of the nuclear pulposus in an intervertebral disc.

FIG. 14 is a detail view of the implant end portion of the assembly of FIG. 13.

FIG. 15 is a perspective view of an implant launcher and fill assembly according to the present invention shown with an introducer sheath, launcher sheath, fill tube positioned prior to deployment of an implant in the nuclear space of an intervertebral disc and with the proximal end portions of the introducer sheath and launcher sheath partially cut away to expose the implant and buttress.

FIG. 16 is a detail view of the implant end portion of the assembly of FIG. 15.

FIG. 17 is a perspective view of the assembly of FIG. 15 after insertion of the implant in the nuclear space of the intervertebral disc.

FIG. 18 is a detail view of the implant end portion of the assembly of FIG. 17.

FIG. 19 a perspective view of the assembly of FIG. 15 after deployment of the implant in the nuclear space and prior to retraction of the implant and inner annular buttress.

FIG. 20 is a detail view of the implant end portion of the assembly of FIG. 19.

FIG. 21 is a perspective view of the assembly of FIG. 15 after partial retraction of the implant and inner annular buttress with the inner annular buttress shown engaging and plugging the annular opening in the intervertebral disc.

FIG. 22 is a detail view of the implant end portion of the assembly of FIG. 21.

FIG. 23 is a perspective view of the assembly of FIG. 15 after the implant is inflated.

FIG. 24 is a detail view of the implant end portion of the assembly of FIG. 23

FIG. 25 is a perspective view of an intervertebral stent in accordance with the present invention.

FIG. 26 illustrates the stent of FIG. 25 in a collapsed configuration.

FIG. 27 is a schematic diagram of the stent of FIG. 25 installed in a nuclear cavity in between two adjacent lumbar vertebrae in accordance with the present invention.

FIG. 28A illustrates a perspective view of an alternative intervertebral stent in accordance with the present invention.

FIG. 28B illustrates a top view of the stent of FIG. 28A.

FIG. 28C illustrates a lateral view of the stent of FIG. 28A implanted between two adjacent cervical vertebrae.

FIG. 28D illustrates an anterior view of the stent of FIG. 28A implanted between two adjacent cervical vertebrae.

FIG. 28E illustrates a superior view of the stent of FIG. 28A in an exemplary orientation with respect to a cervical vertebrae.

FIG. 29 illustrates the stent of FIG. 25 collapsed around bladder-type implant in accordance with the present invention.

FIG. 30 illustrates a cross-sectional view of an implant having an inflatable bladder with a textured surface in accordance with the present invention.

FIG. 31 illustrates a cross-sectional view of an implant having filler material comprising microspheres in accordance with the present invention.

FIG. 32 illustrates a cross-sectional view of an implant having reinforced peripheral walls in accordance with the present invention.

FIG. 33 illustrates a cross-sectional view of an implant having multiple chambers in accordance with the present invention.

FIG. 34 illustrates a top cross-sectional view of the implant of FIG. 33.

FIG. 35 illustrates a cross-sectional view of an alternative implant with a suspended chamber.

FIG. 36 shows a schematic view of a system for repairing an annular defect.

FIGS. 37A illustrates an anchor of the system of FIG. 36 installed in the vertebral body.

FIGS. 37B illustrates a close-up view of the mesh used in the system of FIG. 36.

FIGS. 37C illustrates an exemplary cable tie that may be used in the system of FIG. 36.

### DETAILED DESCRIPTION OF THE INVENTION

In the following descriptive material, various aspects and embodiments of the invention are described as systems, devices or methods. It will be appreciated that these aspects and embodiments can be used in a stand-alone manner, and further that any aspect or embodiment can be used in combination with one or more of the aspects or embodiments described herein. In addition, those skilled in the art will appreciate that any of the aspects or embodiments of the invention described herein can be used in combination with other devices, systems and methods known in the art.

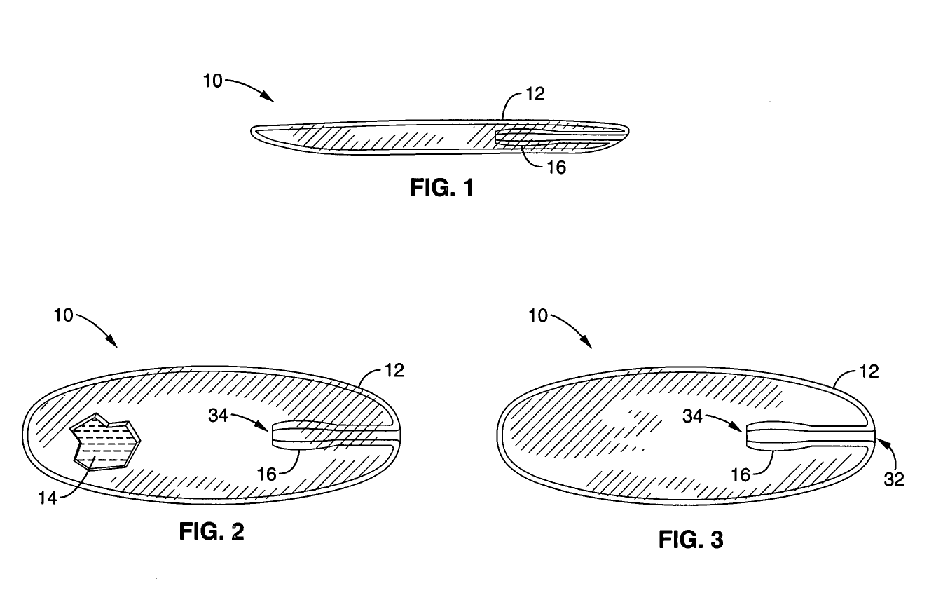

Referring more specifically to the drawings, for illustrative purposes the present invention is embodied in the apparatus generally shown in FIG. 1 through FIG. 37C. It will be appreciated that the apparatus may vary as to configuration and as to details of the parts, and that the method may vary as to the specific steps and sequence, without departing from the basic concepts as disclosed herein.

1. Nuclear Disc Implant

Referring first to FIG. 1 through FIG. 3, an implant 10 according to the present invention comprises a collapsible membrane 12 that is formed into a inflatable balloon or sack that will conform to the shape of the nucleus pulposus when inflated. Membrane 12 preferably comprises an inert material such as silicone or a similar elastomer, or a biodegradable and biocompatible material such as poly (DL-lactic-co-glycolic acid; PLGA). Since the implant will serve as an artificial inner annulus, and its internal chamber will contain a pressurized nuclear filler material 14 used for inflation, the membrane material should be relatively impermeable while possessing the necessary compliance and strength. In addition, the membrane material should be sufficiently flexible so that the implant can easily be passed through a surgical catheter or cannula for insertion.

Table 1 compares certain characteristics of the inner annulus to a number of commercially-available elastomers that were considered for the membrane material. Key design requirements were biocompatibility, stiffness, and elongation-to-failure. While any of these materials, as well as other materials, can be used, our preferred material was aliphatic polycarbonate polyurethane (HT-4) which has a stiffness that closely approximates that of the inner annulus, can be fabricated into complex shapes using dip molding, possess significant failure properties, and has a track-record for in vivo use.

The peripheral surface of the implant is preferably coated with one or more bioactive substances that will promote healing of the inner annulus and integration of the implant with the surrounding annular tissue. Also, the top and bottom surfaces of the implant are preferably coated with one or more bioactive substances that will promote healing of the cartilaginous endplates and integration of the implant with the endplates.

To limit the amount of lateral bulging when the implant is axially compressed, the peripheral surface of the implant can be reinforced with a fiber matrix if desired. In that event, the angle of the fibers relative to the vertical axis of placement should be approximately ±60° to closely approximate that of the native collagen fibers in the inner annulus.

Implant 10 includes an integral, internal, self-sealing, one-way valve 16 that will allow the implant to be inserted in a deflated state and then be inflated in situ without risk of deflation. Valve 16 functions as a flapper valve to prevent leakage and maintain pressurization of the implant when pressurized with the nuclear filler material. Because valve 16 is internal to the implant, compression of implant 10 will place internal pressure on valve 16 to keep it in a closed position. Due to the self-sealing nature of valve 16, the same pressure that might be sufficient to allow the nuclear filler material to escape will cause valve 16 to remain closed so as to create a barrier to extrusion.

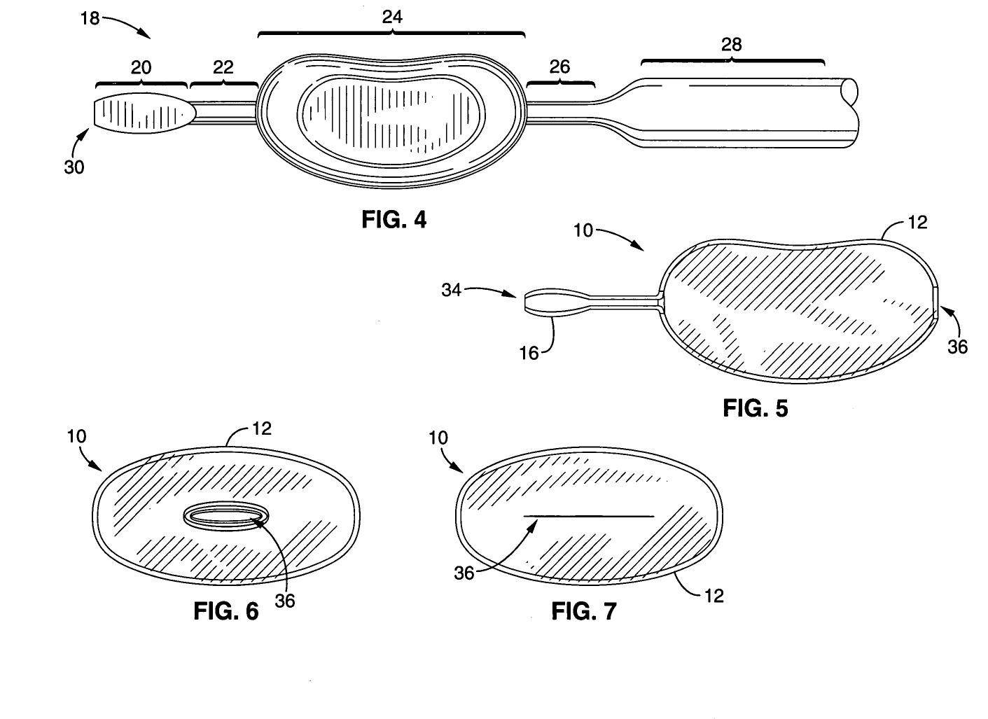

To better understand the operation and configuration of valve 16, reference is now made to FIG. 4 which shows the preferred embodiment of a mandrel 18 for fabricating the implant. Mandrel 18 preferably comprises a planar stem portion 20, a first cylindrical base portion 22, a mold portion 24, a second cylindrical base portion 26, and a shank 28. To fabricate an implant, distal end 30 of the mandrel is dipped in a bath of membrane material to a defined depth which is generally at a point along second base portion 26 and molded to a thickness between approximately 5 mils and 7 mils.

FIG. 5 generally depicts the configuration of the implant after it has dried on the mandrel. However, the mandrel is not shown in FIG. 5 so that the implant can be more clearly seen. After the membrane material dries on the mandrel, it is drawn off of the mandrel by rolling it toward distal end 30. As a result, the membrane is turned inside-out. By inverting the membrane in this manner, the portion of membrane material that coated stem portion 20 becomes valve 16 which is now located inside the implant as shown in FIG. 3. The portion of membrane material that coated first base portion 22 becomes an entrance port 32 into valve 16. Note that the distal end 34 of valve 16 was sealed during molding, while the distal end 36 of the implant is still open as shown in FIG. 5 and FIG. 6. Accordingly, to finish the fabrication process, distal end 36 of the implant is heat-sealed to close it off as shown in FIG. 7.

To inflate the implant, a needle-like fill stem is inserted through entrance port 32 so as to puncture the distal end 34 of valve 16 and extend into the interior chamber of the implant. The implant is then filled with a fluid material, such as a high molecular weight fluid, gel or combination of fluid and elastomer which has a viscosity that will permit its introduction into the implant through, for example, an 18-gauge needle. The specific properties of filler material 14 should allow the material to achieve and maintain the desired osmotic pressure. The filling takes place after the implant is placed within the disc. Preferably filler material is a cross-linkable polyethylene glycol (PEG) hydrogel with chondroitin sulfate (CS) and hyaluronic acid (HA) with or without host cells as will now be described.

Table 2 shows the characteristics of a number of commercially-available hydrogels that were considered for filler material 14. While any of these materials, as well as other materials, can be used, we selected an in situ cross-linkable polyethylene glycol (PEG) gel because of its bio-compatibility and physical properties. The PEG gel is a two component formulation that becomes a low-viscosity fluid when first mixed and which cross-links to a firm gel after insertion. The cross-link time depends on the formulation. A key feature of the gel is its osmotic pressure. We sought to formulate a gel that would possess an osmotic pressure of near 0.2 MPa which is that of the native nucleus pulposus.

The preferred PEG gel comprises a nucleophilic “8-arm” octomer (PEG-NH2, MW 20 kDa) and a “2-arm” amine-specific electrophilic dimer (SPA-PEG-SPA, MW 3.4 kDa), and is available from Shearwater Corporation, Huntsville, Ala. The addition-elimination polymerization reaction culminates in a nitrogen-carbon peptide-like linkage, resulting in a stable polymer whose rate of polymerization increases with pH and gel concentration. The range of pH (approximately 10 for the unmodified gel) and concentration (approximately 0.036 g/mL to 0.100 g/mL) investigated resulted in a polymerization time of approximately 10 minutes to 20 minutes. To fortify the hydrogel's inherent swelling due to hydrogen bonding, high molecular weight additives chondroitin sulfate (CS) and hyaluronic acid (HA) with established fixed charged densities were incorporated into the gel matrix.

The swelling pressures of the hydrogel filler (cross-linked polyethylene glycol (PEG) hydrogels and derivatives incorporating HA and CS) were measured by equilibrium dialysis as a function of gel and additive concentration. Polyethylene glycol (Molecular Weight 20 kDa available from Sigma-Aldrich Corporation) was also used as the osmotic stressing agent, while molecularporous membrane tubing was used to separate sample gels from the dialysate. Gels were formed over a broad concentration range (0.036 to 0.100 g/mL), weighed, placed in dialysis tubing (Spectra/Por Membrane, Molecular Weight Cut Off of 3.5 kDa available from Spectrum Medical Industries), and allowed to equilibrate for 40 to 50 hours in the osmotic stressing solution, weighed again to determine hydration, then oven dried (at 60 degrees Celsius) and weighed once again. Hydration values taken at various osmotic pressures allowed the construction of osmotic pressure curves. By adjusting the concentrations of CS or HA we were able to meet our design criteria, successfully achieving swelling pressures above 0.2 MPa. A potential deleterious interaction between the elastomer and hydrogel was noted. One PEG-CS specimen aged in saline demonstrated breakdown of the elastomer shell. This may have been due to the relatively low-molecular weight CS penetrating into the membrane material (polyurethane) leading to an increased rate of hydrolysis.

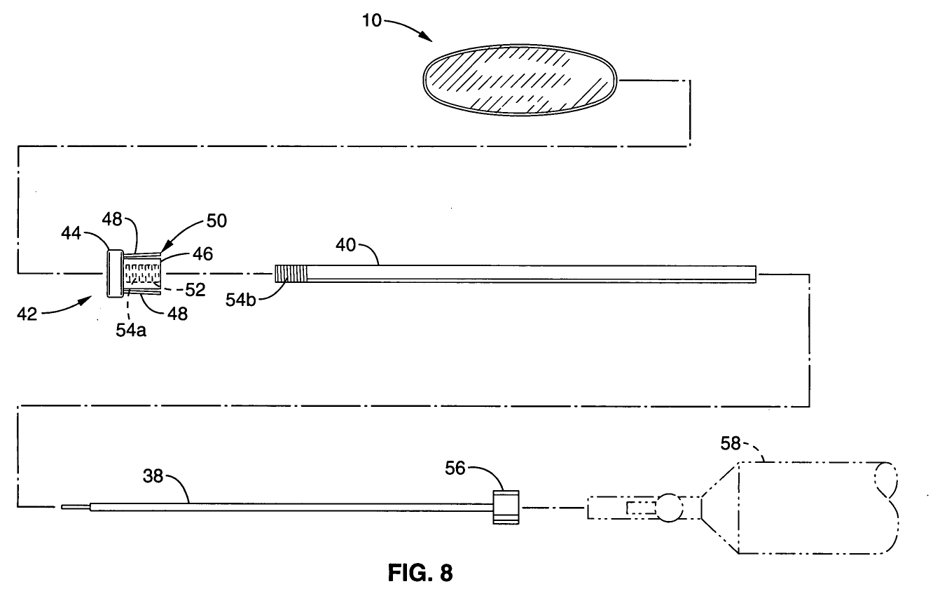

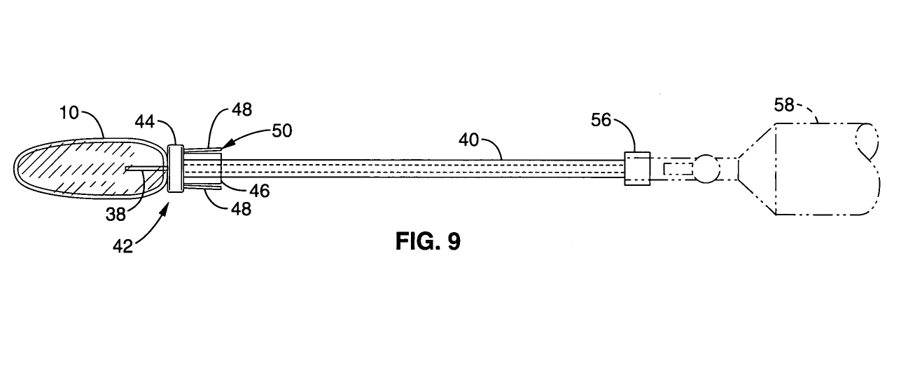

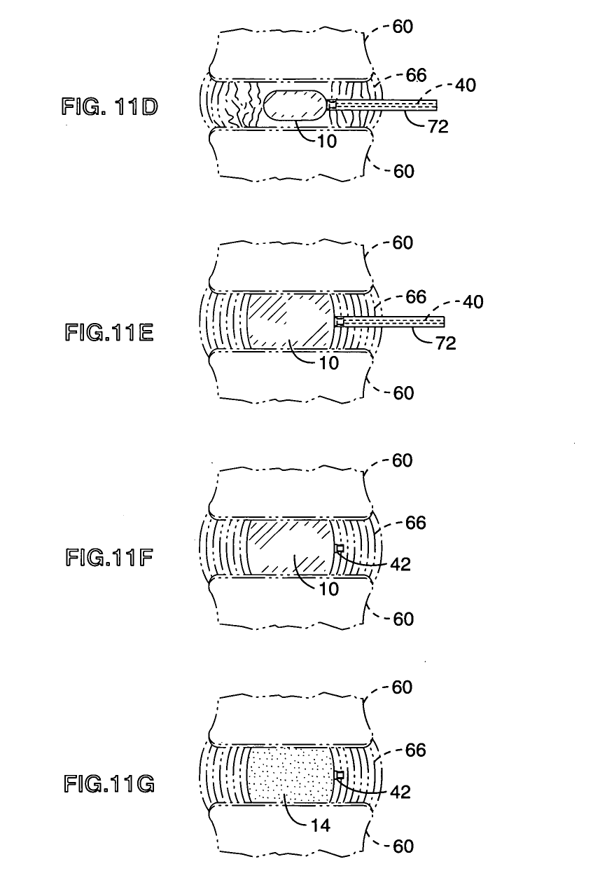

Referring now to FIG. 8 and FIG. 9, the invention includes an implant delivery system comprising a hollow implant fill stem 38, a hollow buttress positioner 40, and an inner annular buttress 42. Implant fill stem 38 is configured for inflating implant 10 after insertion, and inner annular buttress 42 is configured to extend into and block a hole 66 (see FIG. 11A) that is made in the annulus for insertion of implant 10. Once inserted, inner annular buttress 42 prevents extrusion of the implant during spinal loading. Inner annular buttress 42 preferably comprises a polymer head portion 44 of suitable diameter for plugging hole 66, a smaller diameter polymer body portion 46 extending from head portion 44, and metal barbs or pins 48 having ends 50 that extend outward in relation to body portion 46 such that they will engage the annulus to prevent expulsion of inner annular buttress 42 (and implant 10) during spinal loading. Pins 48, which can be formed of stainless steel, Nitinol®, or the like, can be molded or otherwise inserted into head portion 44 for retention therein.

An inner passage 52 extends through inner annular buttress 42 for attachment to buttress positioner 40 and insertion of fill stem 38 through inner annular buttress 42 into implant 10. Inner passage 52, head portion 44 and body portion 46 are preferably coaxial. Buttress positioner 40 and inner annular buttress 42 are coupled together using mating threads 54a, 54b or another form of detachable coupling that allows buttress positioner 40 to be easily removed from inner annular buttress 42 after placement. Note that inner annular buttress 42 can be attached to implant 10 using adhesives, ultrasonic welding or the like, or can be separate and unattached from implant 10.

Fill stem 38 includes a collar 56 for attachment to a syringe 58 or other device to be used for inflating the implant with the filler material. Fill stem 38 and syringe 58 are coupled together using threads (not shown) or another form of detachable coupling. Preferably, syringe 58 includes a pressure gauge (not shown) for determining the proper inflation pressure. The implant and delivery system would be deployed into the nucleus pulposus space by being inserted into a conventional catheter, cannula or the like (not shown) having a retractable cover (not shown) that protects the implant during insertion.

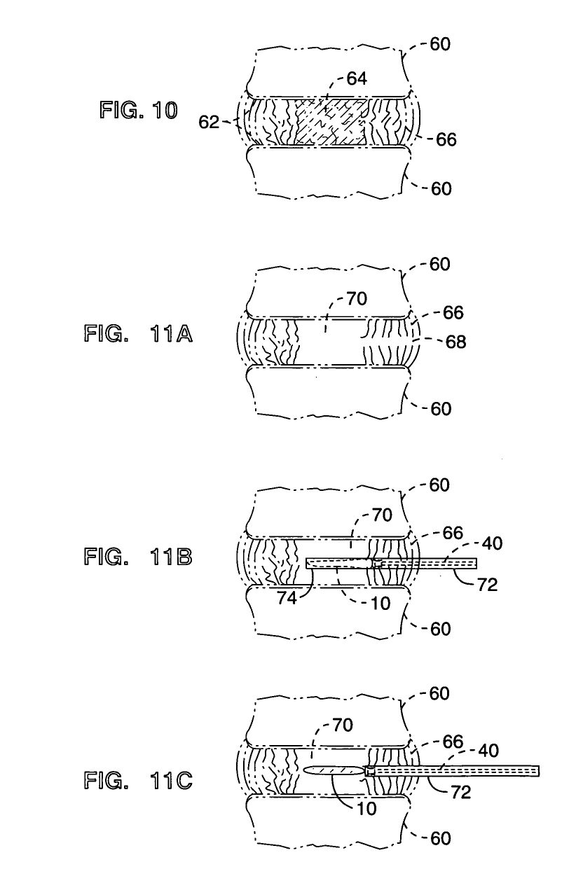

FIG. 10 depicts the vertebral bodies 60, cartilage endplates 62, degenerated nucleus 64, and degenerated annulus 66 in the spine. The indications for use of the implant are a patient with back pain or radiating pain down the leg where the cause of the pain has been determined to be a herniated disc which is impinging on the surrounding spinal nerves. Deployment of the implant is preferably according to the following surgical procedure shown in FIG. 11A through FIG. 11G which is minimally invasive.

As shown in FIG. 11A, the first step in the surgical procedure is to perform a minimally invasive postero-lateral percutaneous discectomy. This is executed by making a small hole 68 through the annulus fibrosus of the intervertebral disc and removing the nucleus pulposus tissue through that hole. Several technologies were considered to facilitate removing degenerated nuclear material through a small opening made through the annulus fibrosus. The most promising technology is the ArthroCare Coblation probe (ArthroCare Spine, Sunnyvale, Calif.). This device vaporizes the nucleus in situ. Because of density differences that exist between the nucleus and annulus, the Coblation probe removes the less-dense nuclear material more easily than the annulus. This allows the surgeon to remove the nuclear material while minimizing damage to the remaining annulus or adjacent vertebral body.

The referred protocol for creating a nuclear space for the implant comprises making a small puncture within the annulus with a pointed, 3 mm diameter probe. This pointed probe serves to separate annular fibers and minimize damage to the annulus. Next, a portion of the nucleus is removed using standard surgical instruments. The Coblation probe is then inserted. Suction and saline delivery are available with the probe, although we have found that suction through another portal using, for example, a 16-gage needle, may be required. A critical feature of device success is the method of creating a nuclear space while minimizing trauma to the outer annulus fibrosus. The outer annulus should be preserved, as it is responsible for supporting the implant when pressurized.

Next, as shown in FIG. 11B, the deflated implant 10 is inserted into the empty nuclear space 70. This is accomplished by inserting the implant through a conventional insertion catheter (cannula) 72. Note that fill stem 38, buttress positioner 40 and inner annular buttress 42 are also inserted through catheter 72, which also results in compression of pins 48. The cover 74 on the insertion catheter 72 is then retracted to expose the implant as shown in FIG. 11C. Next, as shown in FIG. 11D, the implant is inflated with the filler material 14, until it completely fills the nuclear space 70. FIG. 11E shows the implant fully inflated. Note the resultant increase in disc height and restoration of tensile stresses in the annulus. The pressurized implant initiates the restoration of the original biomechanics of the healthy disc by increasing the disc height, relieving the annulus of the compressive load, and restoring the normal tensile stress environment to the annulus. The restoration of the normal tensile stress environment in the annulus will promote the annular cells to regenerate the normal annulus matrix.

The catheter and delivery system (e.g., fill stem 38 and buttress positioner 40) are then removed, leaving inner annular buttress 42 in place and implant 10 sealed in position as shown in FIG. 11F. Note that inner annular buttress 42 not only serves to align and place the implant, but prevents extrusion during spinal loading. In addition, the one-way valve 16 in the implant prevents the hydrogell growth factor mixture from leaking back out of the nucleus implant. Therapeutic agents on the peripheral and top/bottom surfaces of the implant stimulate healing of the inner annulus and cartilage endplates. In addition the surface growth factors will also promote integration of the implant with the surrounding tissue.

Finally, FIG. 11G depicts the implant biodegrading after a predetermined time so as to allow the hydrogell growth factor mixture to play its bioactive role. The hydrogel is hydrophilic and thereby attracts water into the disc. Much like the healthy nucleus pulposus, the hydrogel creates a swelling pressure which is essential in normal disc biomechanics. The growth factor which is included in the hydrogel stimulates cell migration, and proliferation. We expect the environment provided for these cells to stimulate the synthesis of healthy nucleus pulposus extracellular matrix components (ECM). These cells will thereby complete the regeneration of the nucleus pulposus.

It will be appreciated that the implant can be inserted using other procedures as well. For example, instead of performing a discectomy (posterolateral or otherwise), the implant could be inserted into a preexisting void within the annulus that arises from atrophy or other form of non-device-induced evacuation of the nucleus pulposus, such as for, example, by leakage or dehydration over time.

### EXAMPLE 1

Prototype implant shells were fabricated by Apex Biomedical (San Diego, Calif.). The fabrication process included dip molding using a custom-fabricated mandrel. The mandrel was dipped so that the elastomer thickness was between 5 and 7 mils (0.13-0.17 mm). After dipping, the implant was removed from the mandrel, inverted (so that the stem was inside the implant) and heat-sealed at the open end. This process resulted in a prototype that could be filled with the PEG gel, which when cross-linked could not exit through the implant stem. The stem effectively sealed the implant by functioning as a “flapper valve”. This means that by being placed within the implant, internal pressures (that might serve to extrude the gel) compress and seal the stem, creating a barrier to extrusion. This sealing mechanism was verified by in vitro testing.

### EXAMPLE 2

Elastomer bags filled with PEG were compressed to failure between two parallel platens. The implants failed at the heat seal at approximately 250 Newtons force. These experiments demonstrated that under hyper-pressurization, the failure mechanism was rupture at the sealed edge, rather than extrusion of gel through the insertion stem. When the device is placed within the intervertebral disc, support by the annulus and vertebral body results in a significantly increased failure load and altered construct failure mechanism.

### EXAMPLE 3

Ex vivo mechanical testing were performed with human cadaveric spines to characterize the performance of the device under expected extreme in vivo conditions. We conducted a series of experiments that consisted of placing the device in human cadaveric discs using the developed surgical protocols and then testing the construct to failure under compressive loading. The objective of these experiments was to characterize the failure load and failure mechanism. The target failure load was to exceed five times body weight (anticipated extremes of in vivo loading). Importantly, the failure mode was to be endplate fracture and extrusion of the implant into the adjacent vertebra. This is the mode of disc injury in healthy spines. We did not want the construct to fail by extrusion through the annulus, particularly through the insertion hole, since this would place the hydrogel in close proximity to sensitive neural structures.

Load-to-failure experiments demonstrated that the implant may sustain in excess of 5000 N (approximately seven times body weight) before failure, and that the failure mode was endplate fracture. These preliminary experiments demonstrate that the implant can sustain extremes in spinal compression acutely.

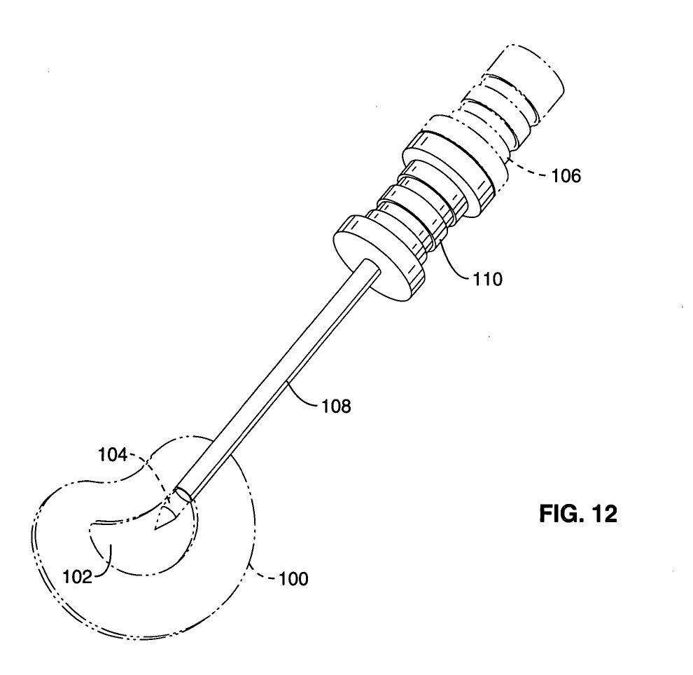

Referring now to FIG. 12, the nuclear space can be prepared for receiving the implant by removing degenerated nuclear material using a coblation probe or the like as described above. Upon exposing the targeted disc 100, the nuclear space 102 can be accessed via a trocar 104, such as a stainless steel, 7 Fr. OD, trocar with a small Ultem handle 106. Preferably, a corresponding 7 Fr introducer sheath 108 also having a small Ultem handle 110, is used for insertion of the trocar. An example of a suitable introducer sheath is a 7 Fr plastic sheath with 0.003 inch walls and a 1.5 inch working length, such as a modified Cook or equivalent. The trocar is then removed upon access leaving a patient access point. Use of an introducer tends to minimize wear and tear on the hole, thus maximizing engagement of inner annular buttress 42. In the embodiment shown, inner annular buttress 42 would typically have a 0.071 OD and a length of 0.070 inches, and carry three pins 48 having a diameter of approximately 0.008 inches and a length of approximately 0.065 inches.

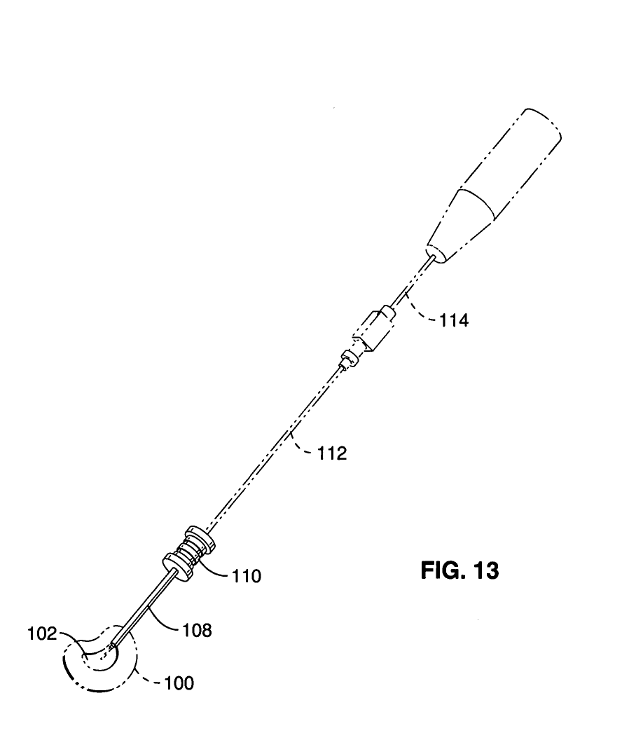

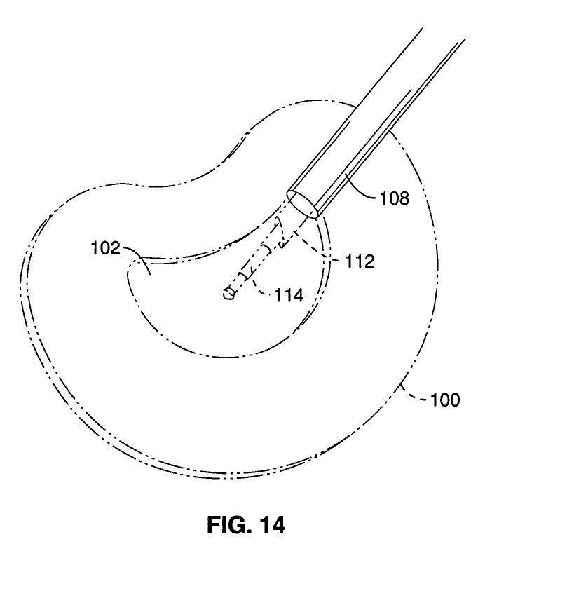

Referring to FIG. 13 and FIG. 14, a Crawford needle 112 (e.g., 17 gage×6 inch, included with the ArthroCare Convenience Pack Catalog No. K7913-010) and ArthroCare Perc-DLE Spine Wand 114 (ArthroCare catalog number K7813-01) are introduced into the nucleus through the introducer sheath 108 and the nucleus pulposus is ablated. By moving the Wand in and out of the needle, the degree of articulation of the distal tip can be controlled. The Crawford needle also provides added rigidity for improved manipulation of the device.

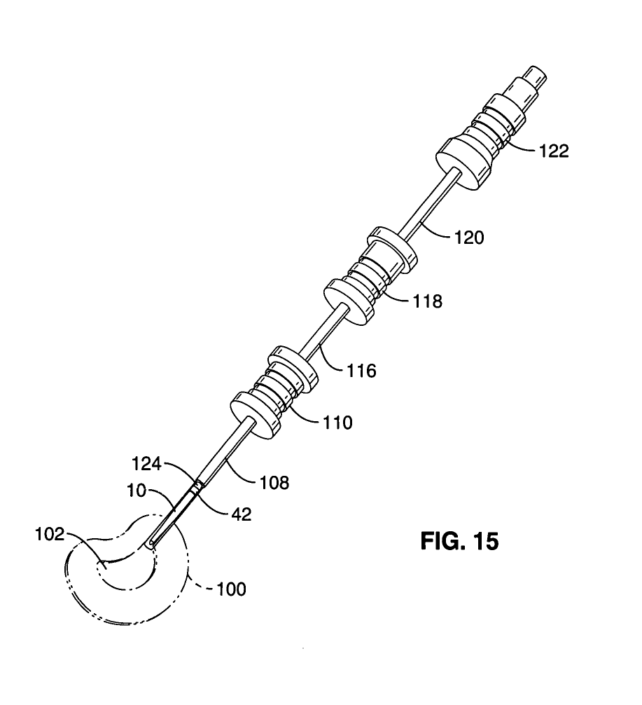

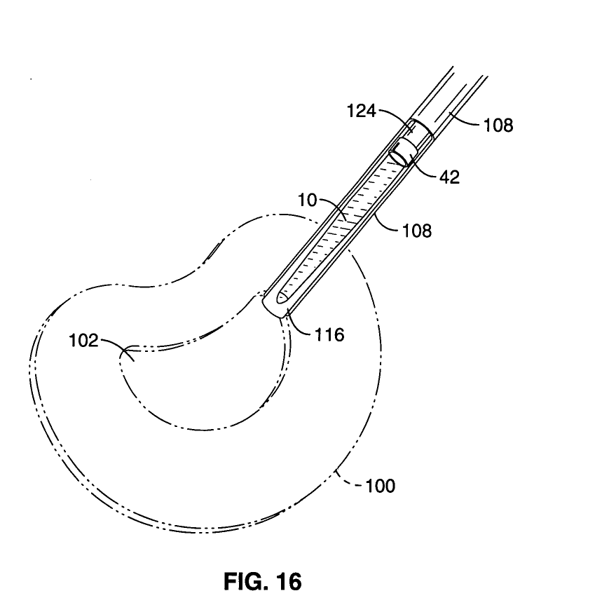

Referring now to FIG. 15 and FIG. 16, an alternative embodiment of the delivery system shown in FIG. 8 and FIG. 9 is illustrated. In this embodiment, introducer sheath 108 is used as a port into the nuclear space 102. In FIG. 15, the end portion of introducer sheath 18 has been cutaway for clarity. A plastic launcher sheath 116 (e.g., 0.084 inch×0.090 inch×3 inch) is slidably insertable into the introducer sheath is provided. Note that the end portion of launch sheath 116 has also been cutaway for clarity. Preferably, launcher sheath 116 includes a small plastic handle 118, and all or a portion of the launcher sheath is preferably flexible to assist with deployment of the implant as described below. A fill tube 120 (e.g., 14 XT×3.9 inch long) is provided that is slidably insertable into launcher sheath 116. Fill tube 120 also preferably includes a small plastic handle 122. The fill tube preferably terminates at its proximal end with a female leur lock 124 having a 0-80 UNF thread to which the assembly of buttress 42 (carrying implant 10) is threadably attached. It will be appreciated that buttress 42 can be attached to leur lock 124 after fill tube 120 has been inserted into launcher sheath 116 and extended therethrough such that leur lock 124 extends through the end of launcher sheath 116. At this point pins 48 can be manually depressed and the un-deployed implant/buttress assembly pulled into the launcher sheath. Alternatively, buttress 42 can be attached to leur lock 124 and fill tube 120 then inserted into launcher sheath 116. With either approach, the assembly of implant 10, buttress 42, launcher sheath 116 and fill tube 120 can then be inserted into introducer sheath 108 and pushed into the nuclear space 102. A small c-clip style spacer or the like (not shown) can be used to maintain separation-between handles 118 and 122 to prevent premature deployment of the implant as will be more fully appreciated from the discussion below.

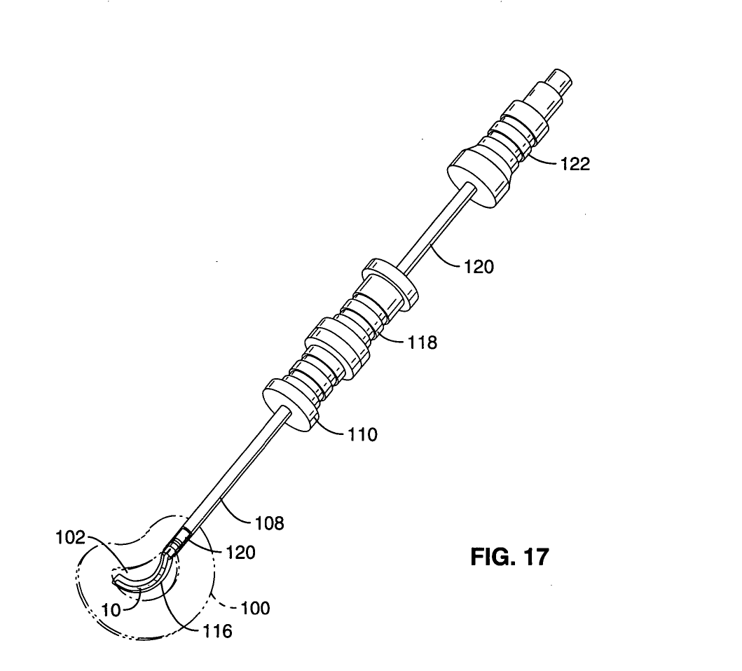

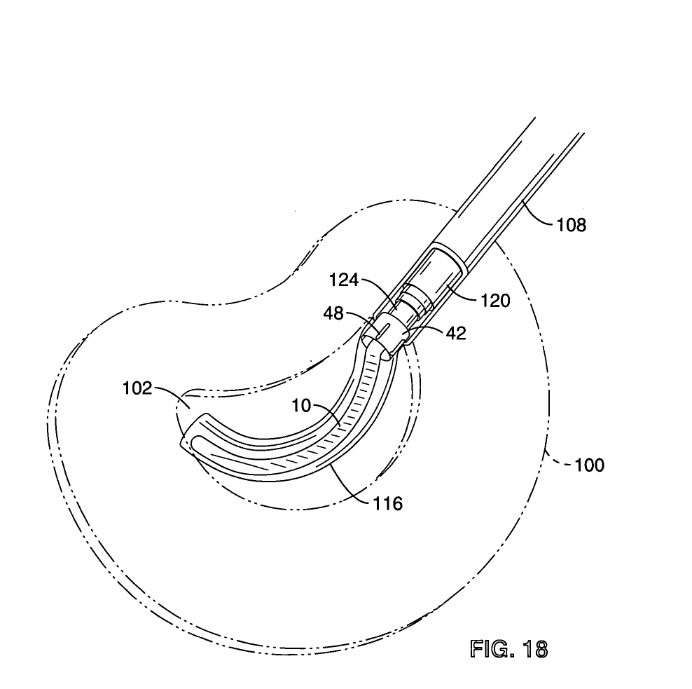

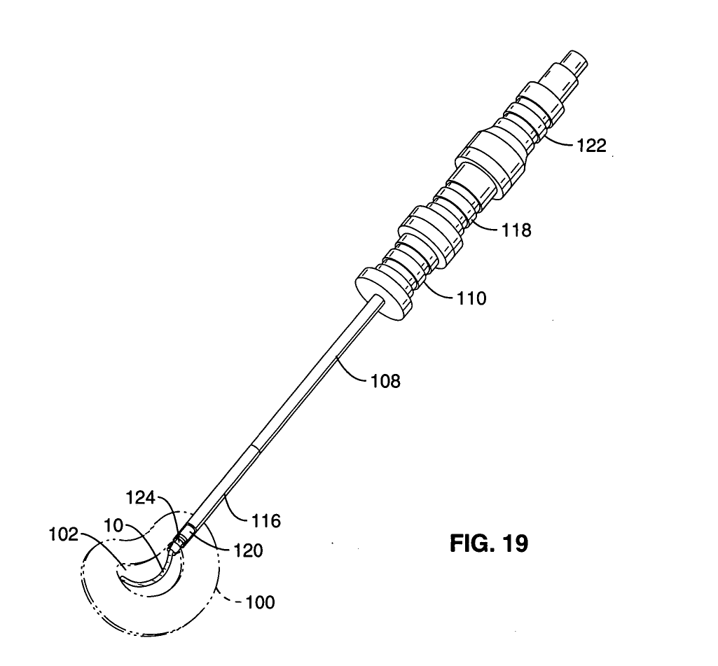

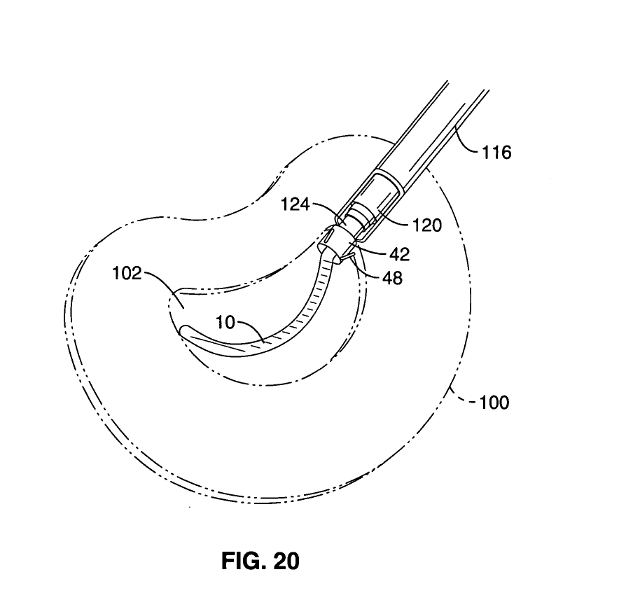

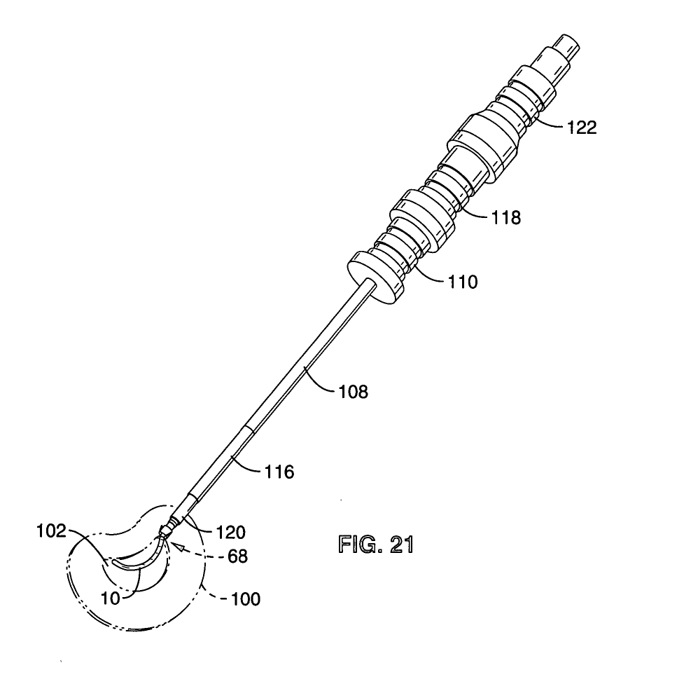

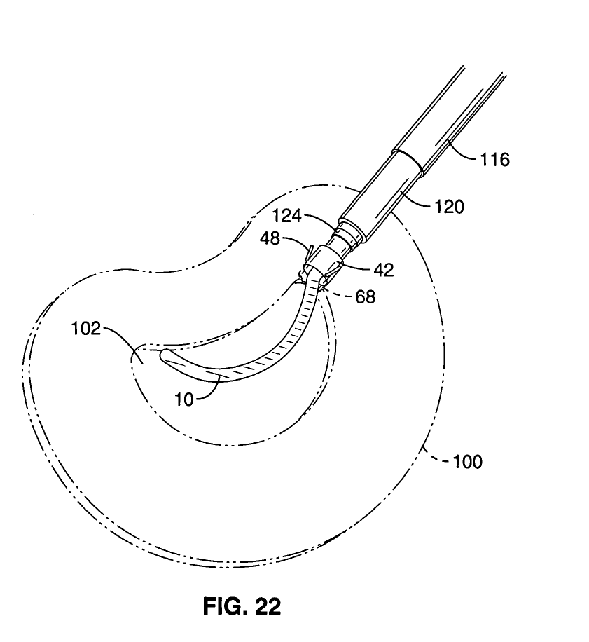

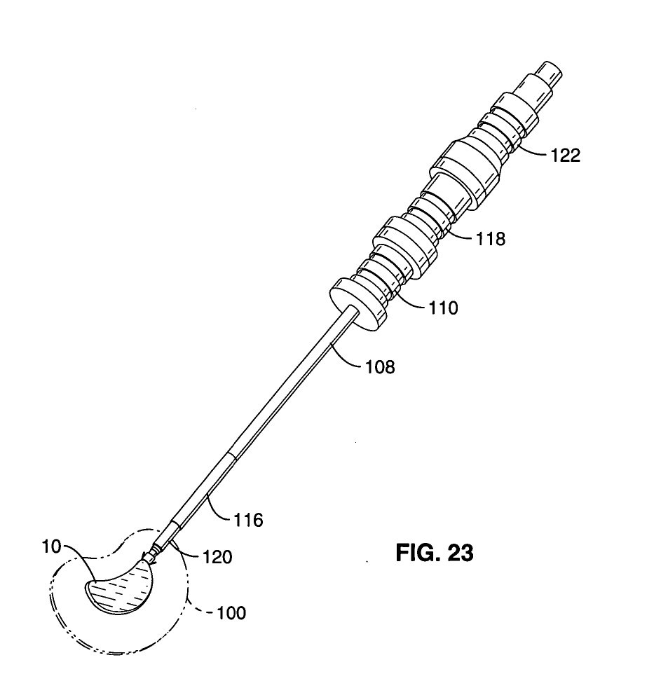

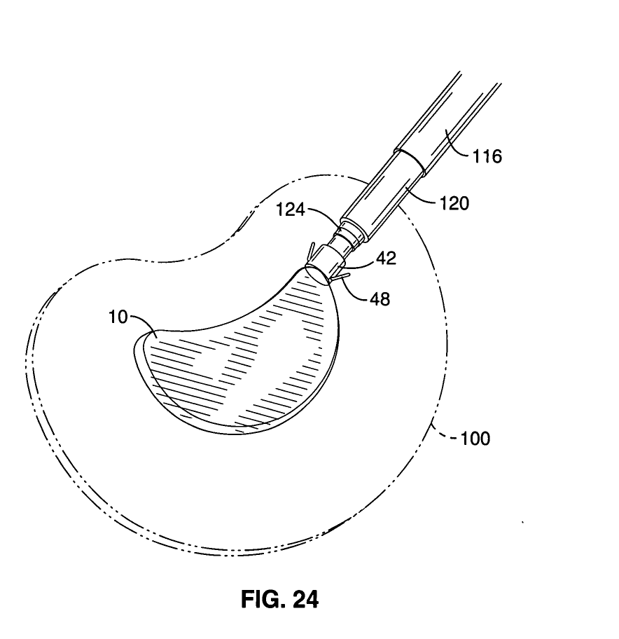

As can be seen from FIG. 17 and FIG. 18, implant 10 can then be advanced into the nuclear space 102 by pushing launcher sheath 116 through introducer sheath 108 until handle 118 contacts handle 110. Note that the flexibility of launcher sheath 106 allows it to deflect if necessary to fit the contour of the nuclear space. FIG. 19 and FIG. 20 then show the implant being deployed by retracting both the introducer sheath 108 and the launcher sheath 116 by pulling handles 110 and 118 back toward handle 122 on fill tube 120 until they are in contact with handle 122. From FIG. 20 it can be seen that pins 48 will then spring outward into the nuclear space and into a position that is ready for engagement with the annulus. Then, as can be seen in FIG. 21 and FIG. 22, pulling back on fill tube 120 will cause the pins 48 on buttress 42 to engage the annulus 68. With inner annular buttress 42 secured in place, implant 10 can then be filled as shown in FIG. 23 and FIG. 24. Once implant 10 is filled, fill tube 120 can be unscrewed from buttress 42 and removed.

2. Intervertebral Stent

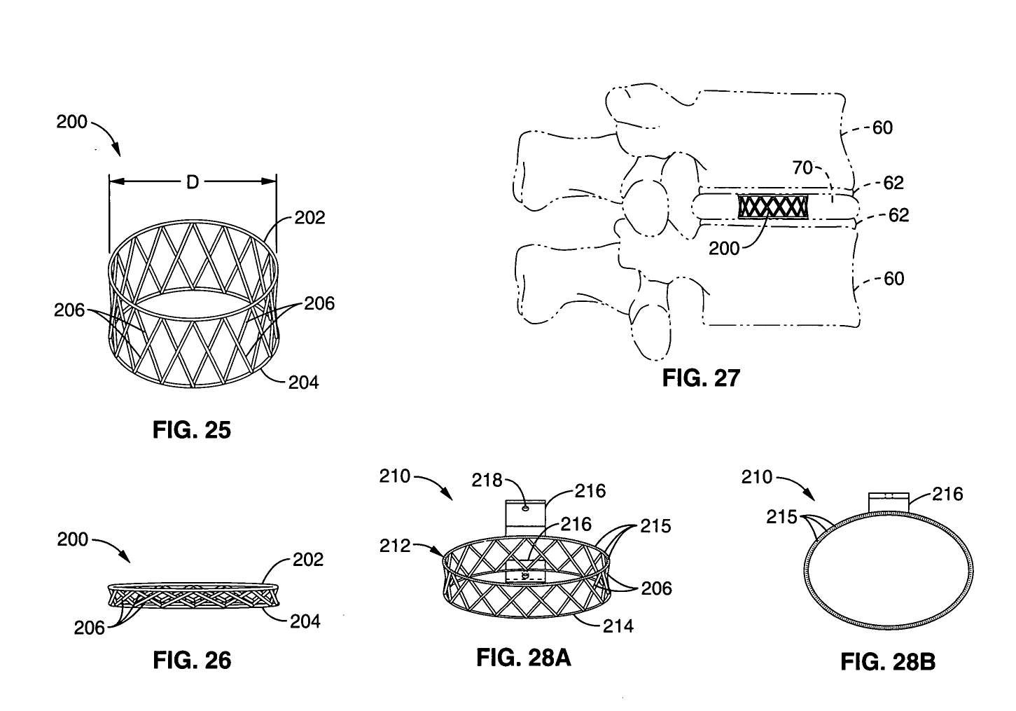

FIG. 25 illustrates an embodiment of the present invention comprising an internuclear stent 200. The stent 200 is configured to keep the nuclear space 70 (shown in FIG. 27 between adjacent lumber vertebra) open by supporting a portion of the intervertebral compression loads and thereby facilitate nuclear regeneration. The stent comprises a top hoop 202 and bottom hoop 204 that are separated by a plurality of lateral members 206. The lateral members 206 and hoops 202, 204 may comprise a memory material or metal, such as a nitinol. The hoops 202, 204 may also be textured to promote bony in growth. The hoops 202, 204 may also have a relatively large gauge to accommodate the higher compressive forces generated in the lumbar spine. The footprint (e.g. diameter D) of the hoops 202, 204 is preferably configured such that the hoops 202, 204 engage with the stiffer peripheral regions of the vertebral endplate 62 while leaving the central endplate open for diffusion into the nucleus. The footprint of hoops 202, 204 may be circular, or elliptical in shape to match virtual cavity 70 produced after nucleus removal.

The sides, or lateral members 206 of the implant 200 are preferably made of flexible nitinol wires that allow the implant to collapse as shown in FIG. 26 to allow for a minimal profile for installation of the stent 200 into the nuclear cavity.

The stent 200 is preferably inserted through an annular portal 68, as shown in FIG. 11A, then expand once in the nuclear cavity 70. Prior to insertion of the stent 200, a minimally invasive postero-lateral percutaneous discectomy removing the nucleus pulposus tissue 64 to create the nuclear cavity 70 as described in the above text associated with FIG. 10A.

The axial stiffness of the stent 200 is preferably only sufficient to partially unload the disc. Thus, the stent 200 is generally not configured to act like a rigid interbody fusion cage, but rather a flexible cage to allow movement while at the same time keeping the nuclear space 70 open for tissue regeneration.

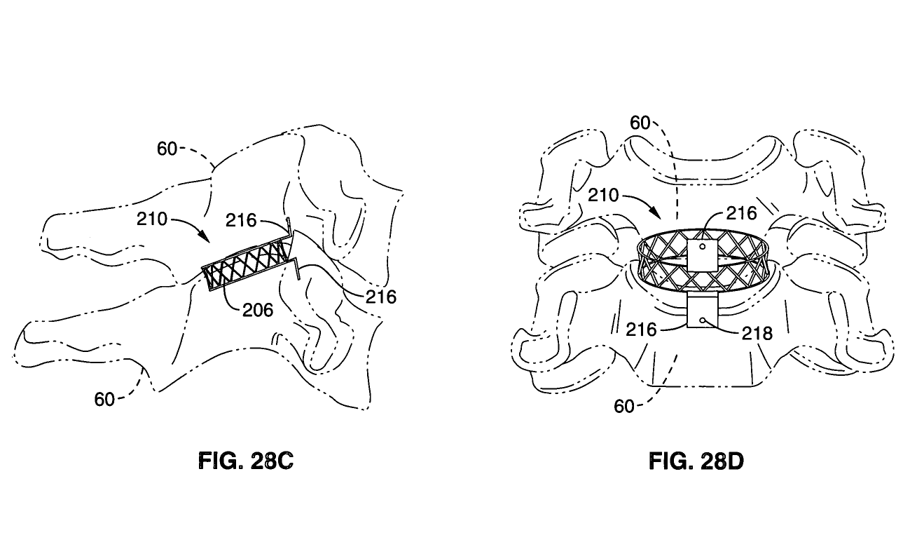

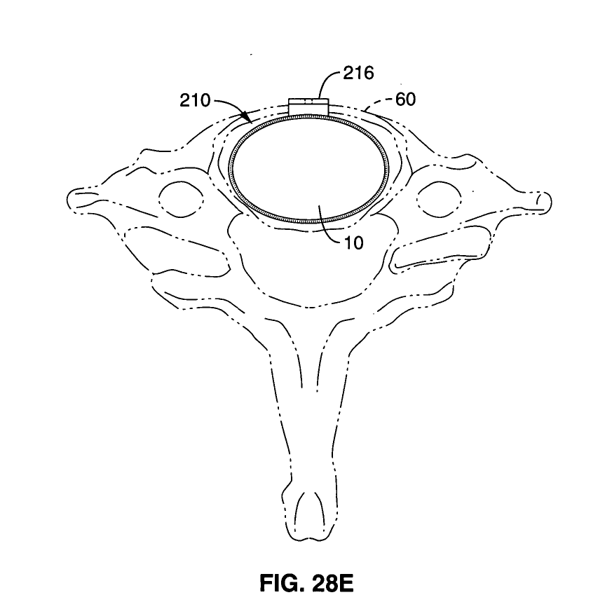

In another embodiment illustrated in perspective view FIG. 28A, stent 210 may be specifically configured to be implanted between adjacent cervical vertebra. As shown in a top view in FIG. 28B, stent 210 is preferably elliptical in shape to match the perimeters of the vertebral bodies. Because treatment of cervical vertebrae often involves removal of much or all of the annulus, the stent 210 preferably has a larger footprint to extend to the perimeter of the vertebral bodies. To help retain the stent 210 from moving with respect to the vertebra, the top hoop 212 and bottom hoop 214 may have serrations 215 to catch the bony vertebral endplate surfaces. Serrations 215 may be in the form of grooves, hook-like protrusions, or a roughed (e.g. bead-blasted) surface to increase friction between the stent 210 and the vertebral bodies 60. For additional retention, the top hoop 212, and/or the bottom hoop 214 may have a flange 216 that extends to the anterior, exterior wall of the vertebral body 60. The flange 216 may have a mounting hole 218 to allow for screw fixation into the anterior wall of the vertebral body 60.

The size, stiffness, and geometry of stents 200, 210 may also be varied to accommodate different patients, or to produce different therapeutic effect. The stents 200, 210 may also be coated with appropriate bioactive factors to facilitate healing, such as TGF-b, FGF, GDF-5, OP-1, or factors that reduce inflammation.

The stent 200, 210, may be a stand-alone device that is used to enhance disc stability while facilitating nuclear regeneration. For example, this stent 200 could be placed after discectomy to facilitate disc repair in a physiologic configuration. The stent may also be used in conjunction with stem cells and polymer carriers to regenerate the nucleus

In an alternative embodiment, the stent 200, 210 may be used to provide additional mechanical support for the biodegradable membrane 10 described in FIGS. 1-24, also described in PCT Application WO 2003/002021, published on Jan. 9, 2003, incorporated herein by reference in its entirety. FIG. 28E illustrates the membrane 10 disposed within stent 210 with respect to the cervical vertebrae body. The stent 210 supports the peripheral expansion of the bladder 10 and holds it in place. This is particularly beneficial in cervical vertebrae implants where most of the host annulus (which would otherwise provide lateral support for the bladder 10) is removed. Thus the membrane 10 generally supports spine compressive loads, while the stent 210 prevents membrane 10 migration or lateral expansion.

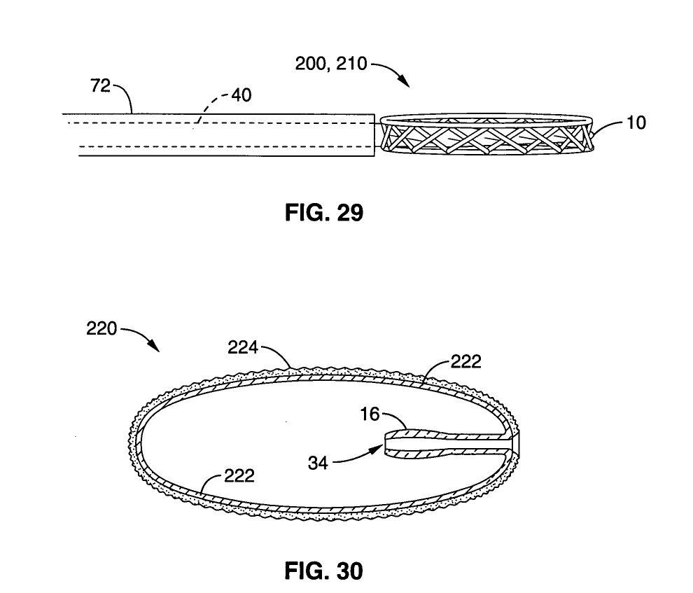

As shown in FIG. 29, the stent 200, 210 may be placed in a collapsed position over deflated membrane 10, and then inserted into the nuclear cavity via insertion catheter (cannula) 72. Once the target region is reached, the membrane 10 may be inflated with filler material, thereby releasing the stent 200,210 from its collapsed state into its expanded state.

Alternatively, the stent 200, 210 may be placed into the nuclear space 70 in its collapsed state by itself, as shown in FIG. 27. Subsequently, after the stent 200, 210 is expanded in the nuclear space, the membrane may be inserted (as shown in FIGS. 11A-11C) into the nuclear space 70 between the upper and lower loops 202, 204 of the stent 200.

The stent of the present invention is particularly advantageous, since no interdiscal stent exists that could work synergictically with surrounding tissues while providing space and the appropriate mechanical environment to facilitate disc regeneration.

3. Surface Texturing as a Means to Stabilize a Nuclear Implant

In a further embodiment of the invention, the surface of the nuclear implant 10 described in FIGS. 1-24 could be textured using a foaming agent along with a lower viscosity formulation of the polyurethane to formulate an enhanced implant 220, as illustrated in FIG. 30.

As a final stage of dip manufacturing, the implant may be dipped into a foamed, uncured polyurethane, forming a final textured surface finish, or layer 224 outside of membrane 222. The final surface texture of the outside layer 224 would typically have an average pore size in the range of approximately 400 microns to approximately 800 microns, volume porosity in the range of approximately 75% to approximately 80%, and thickness of approximately 1 mm to approximately 2 mm. This texturing would facilitate fibrous tissue ingrowth.

The above process may be used to augment mechanisms to stabilize the nuclear implant described above in FIGS. 1-24. The texturing may also be a means to provide growth factors to encourage tissue encapsulation. For example, the implant 220 could be dipped into a growth factor solution prior to implantation. Alternatively, the growth factor could be bound to the textured surface 224.

It is further appreciated that the above described texturing could be also used in combination with other implants known in the art, both in spinal applications, and in other anatomical locations where promoting in growth with surrounding tissues is desirable.

4. Nuclear Implant Filler with Microbubbles/Microspheres

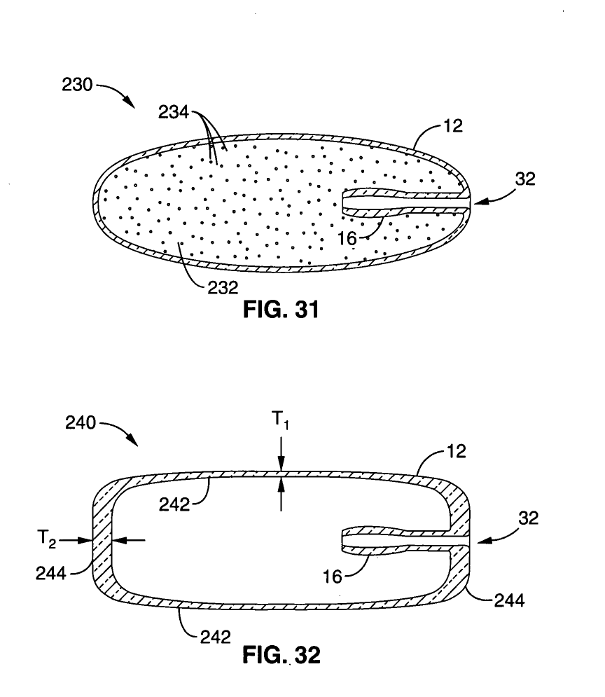

Referring now to FIG. 31, small microbubbles or microspheres 234 could be incorporated into the gel filler 232 of implant 230 (or implant 10 shown in FIGS. 1-24). The microspheres 234 may be gas filled to provide a measure of compliance. The microspheres 234 may also be liquid filled to serve as a reservoir of hydration to help maintain gel hydration over the long term. The chemistry and/or geometry of the bubbles microspheres are configured such that the movement of fluid between microspheres 234 and hydrogel 232 is ‘dynamic’ and dependent on factors such as hydrogel pressure or hydration. For example, it may be of benefit for microspheres 234 to give off water when the hydrogel pressure is high, as a means to maintain implant volume (since high pressure may tend to cause hydrogel to give off water to the external environment).

In an alternative embodiment, the microspheres 234 may serve as a reservoir for drugs having appropriate bioactive factors to facilitate healing to further enhance the performance of the gel filler 232.

It is further appreciated that the microspheres 234 may be used for any inflatable implant currently used in the art.

5. Nuclear Implant Bladder with Peripheral Reinforcement

FIG. 32 illustrates an implant 240 in accordance with the present invention having peripheral reinforcement. For example, top and bottom walls 242 may have the same thickness T₁ as bladder 10 shown in FIGS. 1-24.

Accordingly, side, or peripheral walls 244 may have a different thickness T₂ around the circumference of the bladder. The periphery, or lateral margins 244 of the bladder 240 may be fabricated with a thickened region T₂ to provide localized stiffness.

This increased peripheral thickness may have several beneficial effects, including preventing extrusion, or increasing fatigue resistance. This thickened peripheral edge 244 may also serve to provide device stiffness in an “under inflation situation”. The peripheral thickening may further be configured to cause nonlinearity in overall device stiffness, such as during extreme bending or compression, that would improve overall intervertebral stability. It will be appreciated that an advantage of this aspect of the invention is that peripheral stiffness will enhance mechanical performance.

This dual thickness construction may be incorporated in bladders having the self-sealing internal valve 16 of the present invention, as well as other implant bladders known in the art.

6. Nuclear Implant Bladder With Multiple Chambers

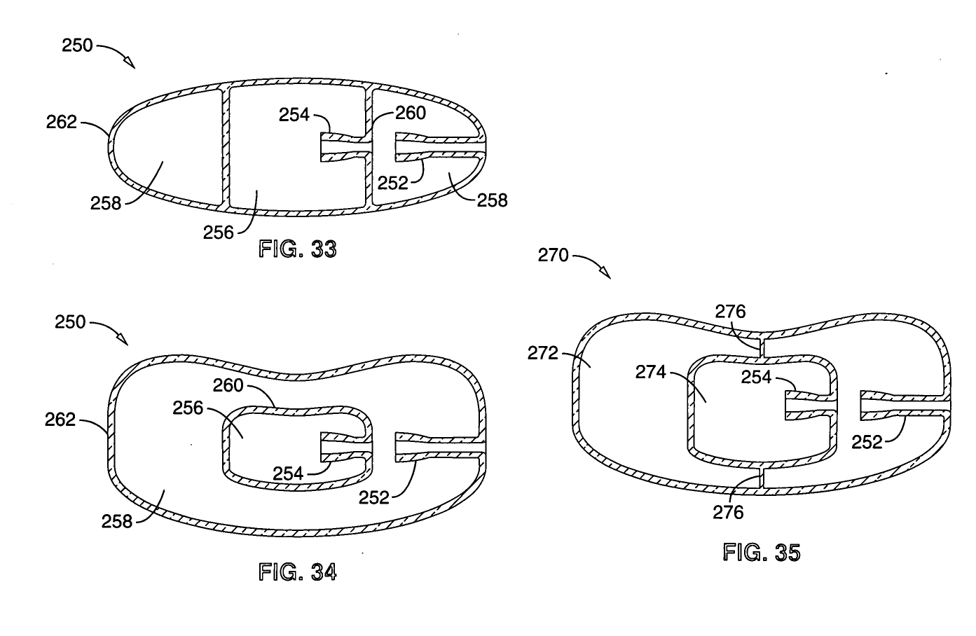

Referring now to FIGS. 33 and 34, implant 250 may be manufactured to have multiple chambers instead of a single bladder. For example, implant 250 may have an internal chamber 256 positioned at the center of the implant, and a peripheral chamber 258 surrounding internal chamber 256, as shown in side cross-section view in FIG. 33, and top cross-section view in FIG. 34.

To facilitate filling of the chambers, implant 250 may have a peripheral valve 252 allowing access to the peripheral chamber 258, and a central valve 254 allowing access to internal chamber 256. Valves 252 and 254 are preferably concentric located with respect to each other, as shown in FIGS. 33 and 34. This facilitates delivery of the inflation medium to both chambers via the same annular portal 68 (shown in FIG. 11A) without having to reposition the implant 250. Alternatively, the valves may be placed at differing locations

Valves 252 and 254 are also preferably integrated, internal, self-sealing valves as shown and described in FIGS. 1-24. However, a 2-piece valve bladder system, or any other bladder/valve configuration known in the art, may be used for the multi-chamber implant of the present invention.

In an alternative embodiment, either or both of the internal and peripheral chambers of implant 250 may also further be divided into a plurality of smaller chambers.

The bladders of implant 250 may also be configured to have differing stiffness. For example, the internal chamber 256 may be filled at a different pressure than the peripheral chamber 258. Additionally, the central chamber 256 may be filled with a softer gel, while the peripheral 258 chamber is filled with a stiffer gel. External walls 262 encasing the peripheral chamber may also have differing or larger thickness than the internal walls 260 of the internal chamber 260. Any of these configurations may be used to advantageously prevent occurrence of implant extrusion through an annular defect.

Finally, the implant 250 could be configured to have an inner mechanical support bladder in chamber 256, and an outer drug delivery bladder in peripheral chamber 258. Thus, the internal chamber 256 may be filled first with a hydrogel having properties that allow the chamber to reach the desired osmotic or swelling pressure, and then the outer chamber 258 is then filled with a liquid or gel carrying therapeutic agents. Potential drugs for delivery include tgf-b and gdf-5 to encourage tissue ingrowth and implant stability. Other choices include, anti-inflammatory drugs to specifically target pain, such as Remicade (anti-tnf-alpha), or glucosamine.

In an alternative version shown in FIG. 35, implant 270 comprises an internal chamber 274 suspended inside peripheral chamber 272. To maintain the central position of the internal chamber 274 with respect to the peripheral chamber 272, supports 276 may connect the two chambers while still allowing the filler material to occupy the internal chambers of the implant.

The multiple bladder approach shown above also has the additional advantage of providing redundancy to the system. Separate chambers may act as a failsafe mechanism in the event that a single bladder fails. In this situation, the multiple bladders would prevent catastrophic failure, with the remaining bladder or bladders maintaining implant performance.

7. Method of Sealing or Repairing the Annulus Fibrosus

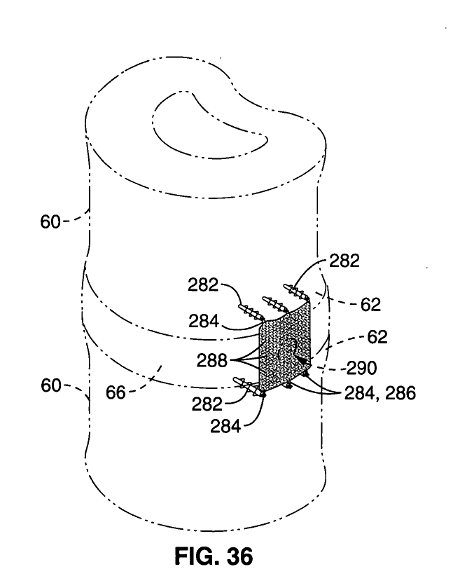

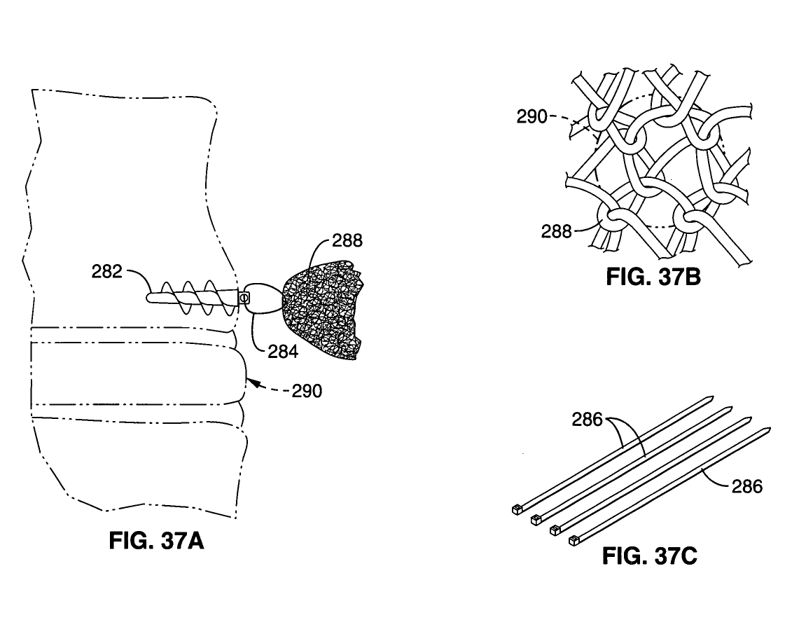

FIG. 36 illustrates a system 280 and method for annular repair (e.g. such as a annular portal 68 generated from an implant as described in the embodiments above, or a region of degenerated annulus) in accordance with the present invention. As illustrated in FIG. 36, one or more suture anchors 282 are first placed into vertebral rims 62 of opposing vertebral bodies 60 (also shown in cross-section view in FIG. 37A). The number of anchors may vary depending on the size of the repair to the annulus 66. The anchors may be installed using a tool (not shown) that allows them to be placed simultaneously. For example, for 3 anchors on each vertebral rim, a pliers-type tool may be used with three tangs on each side (one for each suture anchor 282), each tang having a suture anchor 282 attached. The surgeon could open or close the pliers to accommodate different disc heights.

Once the anchors 282 are set, netting 282 (such as the cargo net 288 shown in FIG. 37B) is attached to the anchors 282 via sutures 284. The cargo-net 288 is made of a woven mesh or fabric, which has a cross-ply that matches the annular architecture. One side of mesh (that is placed against the annulus tissue 66 may comprise a woven polymer such as polyethylene or polypropylene to promote tissue ingrowth (e.g. 800 micron pore size). Correspondingly, the opposite side (placed facing away from the annulus 66), may comprise a woven Teflon, or similar lubricant, to prevent adhesion.

The netting 288 is then stretched over the annulus defect 290, and the free-ends of the sutures 284 are pulled to adjust the fit of the netting 288. This may be facilitated using a ‘cable-tie’ type fastener 286(in addition to, or in lieu of sutures 284), illustrated in further detail in FIG. 37C. The system 280 allows the netting 288 to give during intervertebral movement, thus not unduly constraining the patients natural range of motion, nor unduly stressing the anchor points.

In one embodiment, one of several surgical sealants known in the art may be placed between the mesh 288 and the outer annulus 66.

As an alternative using suture anchors, the surgeon may instead suture directly through and around the vertebral rims 282.

In some instances, the vertebral bodies 60 may be avoided altogether, and sutures 284 may be installed directly through the annulus 66. This may be facilitated using minimally-invasive suturing techniques similar to those currently employed for rotator cuff repair. For example, Opus Medical (www.opusmedical.com) describes an ‘AutoCuff System’ that includes a tool and technique for automated tissue suturing through a narrow/deep tissue channel (this constraint will likely accompany most disc repair surgical techniques). A similar device may be configured for suturing the annulus fibrosus, having customized tips and implant anchors that optimize the repair strength for the disc.

It is appreciated that system and methods illustrated in FIGS. 36A and 37A-C may be used as a stand-alone technology to seal an annular defect after discectomy. Alternatively, the system may be used to “finish up” insertion of a nuclear implant by sealing the annular defect.

It is appreciated existing annular repair approaches attempt to attach to annulus only. Since the quality of the annulus in many cases may be poor, these methods have a high possibility of failure. With the present invention, repair is facilitated by attaching to the vertebral margins in a manner similar to the natural annulus. The approach of the present invention is expected to provide better sealing ability, particularly in situations when the annulus is weakened.

Although the description above contains many details, these should not be construed as limiting the scope of the invention but as merely providing illustrations of some of the presently preferred embodiments of this invention. For example, collagen could be used instead of polymer, and polylysine or type 2 collagen with a cross-linking agent could be used instead of hydrogel. Therefore, it will be appreciated that the scope of the present invention fully encompasses other embodiments which may become obvious to those skilled in the art. In the appended claims, reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

TABLE 1
Elastomer Properties

| | | | | | Tensile | Tensile | |
|---|---|---|---|---|---|---|---|
| | | | Modulus | Modulus | strength | Strength | Elongation |
| Material | Description | Supplier | (psi) | (MPa) | (psi) | (MPa) | (%) |
| Inner | | | | 5 to 10 | | 1 to 3 | 10 to 20 |
| Annulus | | | | | | | |
| HT-3 | aliphatic | Apex | 295.00 | 2.03 | 5300.00 | 36.54 | 470.00 |
| | polycarbonate | Medical | | | | | |
| | polyurethane | | | | | | |
| HT-4 | aliphatic | Apex | 990.00 | 6.83 | 7100.00 | 48.95 | 375.00 |
| | polycarbonate | Medical | | | | | |
| | polyurethane | | | | | | |
| HT-6 | polycarpralactone | Apex | 290.00 | 2.00 | 5800.00 | 39.99 | 850.00 |
| | copolyester | Medical | | | | | |
| | polyurethane | | | | | | |
| HT-7 | aromatic polyester | Apex | 340.00 | 2.34 | 9000.00 | 62.06 | 550.00 |
| | polyurethane | Medical | | | | | |
| HT-8 | aliphatic polyether | Apex | 290.00 | 2.00 | 5500.00 | 37.92 | 710.00 |
| | polyurethane | Medical | | | | | |
| HT-9 | aromatic polyester | Apex | 550.00 | 3.79 | 7000.00 | 48.27 | 550.00 |
| | polyurethane | Medical | | | | | |

TABLE 2
Osmotic Pressure as a Function of Gel Formulation

| Gel Formulation | [PEG] | [HA] | [CS] | Π (MPa) |
|---|---|---|---|---|
| 1 | 3.6% | 0.11% | — | 0.011 |
| 2 | 5.0% | — | — | 0.025 |
| 3 | 5.0% | — | 0.68% | 0.028 |
| 4 | 6.0% | — | — | 0.033 |
| 5 | 7.5% | — | — | 0.052 |
| 6 | 7.5% | 2% | — | 0.080 |
| 7 | 7.5% | — | 6% | 0.130 |
| 8 | 7.5% | 3% | — | 0.155 |
| 9 | 7.5% | — | 11% | 0.220 |
| 10 | 9% | — | 13% | 0.310 |
| 11 | 10% | — | 15% | 0.332 |

The additives in formulation #8 consisted of a pre-swollen HA-PEG gel that was dried then finely cut and incorporated into a new PEG gel.

1. A stent for facilitating regeneration of an intervertebral nucleus, said intervertebral nucleus bounded at its upper and lower extremities by opposing vertebral endplates of adjacent vertebrae, and at its periphery by annulus fibrosus, comprising:
top and bottom portions comprising metal hoops;
said top and bottom portions having a footprint adapted to engage with peripheral regions of the opposing vertebral endplates while leaving a central region of the vertebral endplates open; and
a plurality of lateral members connecting said top and bottom portions;
said lateral members and top and bottom portions configured to allow the stent to collapse for insertion between the adjacent vertebrae;
wherein the stent is configured to expand upon placement between the adjacent vertebrae.

2. A stent as recited in claim 1, wherein the stent, in the expanded configuration, is configured to support at least a portion of compression loads generated between the opposing vertebral endplates to facilitate regeneration of the intervertebral nucleus.

3. A stent as recited in claim 2, wherein the stent functions as a flexible cage to allow movement of the vertebral endplates while at the same time keeping the intervertebral nucleus open for tissue regeneration.

4. A stent as recited in claim 1, wherein the footprint of the top and bottom portions is circular.

5. A stent as recited in claim 1, wherein the footprint of the top and bottom portions is elliptical to match the anatomy of the intervertebral nucleus.

6. A stent as recited in claim 1, wherein the metal hoops and lateral members comprise nitinol.

7. A stent as recited in claim 1, wherein the hoops are textured to promote bony in growth.

8. A stent as recited in claim 7, wherein texturing comprises growth factor to further promote bony in growth.

9. A stent as recited in claim 2, wherein the stent is configured to be expanded around an inflatable membrane.

10. A stent as recited in claim 1:
wherein the stent is configured to be inserted between adjacent lumber vertebrae;
wherein the stent is inserted in a cavity defined by the intervertebral nucleus; and
wherein the stent is shaped to conform to a perimeter of said cavity.

11. A stent as recited in claim 1:
wherein the stent is configured to be inserted between adjacent cervical vertebrae; and
wherein the stent is shaped to extend through a region of removed annulus fibrosus to a perimeter of the vertebral endplates.

12. A stent as recited in claim 11, wherein the top and bottom portions are serrated to engage the vertebral endplates.

13. A stent as recited in claim 11, wherein at least one of the top and bottom portions have an external flange to allow the stent to be fastened to an exterior wall of the vertebrae.

14. A method for facilitating regeneration of the intervertebral disc, the intervertebral disc having a region of nucleus pulposus tissue surrounded by annulus fibrosus, the intervertebral disc disposed between vertebral endplates of adjacent vertebrae, comprising:
inserting a collapsed stent into a nuclear cavity in the nucleus pulposus tissue; and
expanding the stent to support at least a portion of intervertebral compression loads and thereby facilitate nuclear regeneration.

15. A method as recited in claim 14, wherein inserting a collapsed stent into the nuclear cavity comprises:
creating an annular portal in the annulus fibrosus to access the nucleus pulposus;
removing the nucleus pulposus tissue to create the nuclear cavity; and
inserting the collapsed stent through the annular portal and into the nuclear cavity.

16. A method as recited in claim 14, wherein expanding the stent comprises:
expanding upper and lower metal hoops to engage the vertebral endplates;
the upper and lower metal hoops being connected by a plurality of lateral members; and
generating an axial force on the vertebral endplates via a loading from the plurality of lateral members to separate the upper and lower hoops against the endplates.

17. A method as recited in claim 16, wherein the upper and lower metal hoops engage peripheral regions of the vertebral endplates while leaving a central endplate open.

18. A method as recited in claim 14, further comprising:
inserting an inflatable membrane into a nuclear cavity in the nucleus pulposus tissue; and
expanding the inflatable membrane to further support a portion of intervertebral compression loads and thereby facilitate nuclear regeneration.

19. A method as recited in claim 18:
wherein the stent is inserted into a nuclear cavity while in a collapsed configuration over the inflatable membrane; and
wherein expanding the stent comprises inflating the inflatable membrane to release the stent from the collapsed configuration to the expanded configuration.

20. A method as recited in claim 14, wherein the stent functions as a flexible cage to allow movement of the vertebral endplates while at the same time keeping the nuclear cavity open for tissue regeneration.

21. A method for treating an intervertebral disc, the intervertebral disc having a region of nucleus pulposus tissue surrounded by annulus fibrosus, the intervertebral disc disposed between vertebral endplates of adjacent vertebrae, comprising:
inserting a collapsed stent into a cavity in the intervertebral disc; and
expanding the stent to support at least a portion of intervertebral compression loads and thereby facilitate treatment of the disc.

22. A method as recited in claim 21, wherein inserting a collapsed stent into a cavity comprises:
creating an annular portal in the annulus fibrosus to access the nucleus pulposus between adjacent lumbar vertebrae;
removing the nucleus pulposus tissue to create the cavity; and
inserting the collapsed stent through the annular portal and into the cavity.

23. A method as recited in claim 21, wherein inserting a collapsed stent into a cavity comprises:
removing the nucleus pulposus tissue and at least a portion of annulus fibrosis to create a cavity between adjacent cervical vertebrae; and
inserting the collapsed stent into the cavity.

24. A method as recited in claim 21, further comprising:
fastening the stent to an exterior wall of at least one of the adjacent vertebrae.

25. A method as recited in claim 21, further comprising:
inserting an inflatable membrane into the cavity; and
expanding the inflatable membrane in between the stent to further support a portion of intervertebral compression loads.

26. A method as recited in claim 25:
wherein the stent is inserted the cavity while in a collapsed configuration over the inflatable membrane; and
wherein expanding the stent comprises inflating the inflatable membrane to release the stent from the collapsed configuration to the expanded configuration.

27-70. (canceled)
